(19) United States
(12) Patent Application Publication
(10) Pub. No.: US 2012/0184942 A1
(43) Pub. Date: Jul. 19, 2012

(54) COAXIAL STIFFENER FOR DRAINAGE CATHETER

(76) Inventor: Raymond J. Lareau, Westford, MA (US)

(21) Appl. No.: 13/009,659

(22) Filed: Jan. 19, 2011

(51) Int. Cl. A61M 27/00

(52) U.S. Cl. 604/540

(57) ABSTRACT

A co-axial two-piece stiffener for use with an indwelling percutaneous catheter during insertion and removal of the catheter. The stiffener comprises inner and outer stiffener members with the inner member having a large diameter portion with an unconstrained diameter that is the same as or larger than the diameter of the outer stiffener member and a reduced diameter when constrained by the outer stiffener member that is smaller than the diameter of the outer stiffener member.

### FIELD

The embodiments presented herein relate generally to indwelling drainage catheters, and more particularly, to a coaxial stiffener to be used in concert with the insertion of a percutaneous drainage catheter.

### BACKGROUND

Flexible catheters are used for percutaneous drainage of an abscess or pocket of fluid in the body to the exterior by means of gravity or negative pressure. Fluid collection may be the result of an infection, surgery, trauma or other causes. Typical fluids include biliary, nephrostomy, pleural, urinary, and mediastinal collections. As an alternative to providing drainage, these catheters can also be used to introduce substances, such as fluids, into a patient's body.

Successful procedures involving percutaneous drainage depend upon the initial placement of the drainage catheter and having the catheter remain in place for the duration of the treatment. Without adequate anchoring or support, catheter dislodgment may result due to body movements by the patient or under other conditions.

Typically, this can be done by forming a restraining portion in the distal end of the catheter in the form of a pigtail or “J-curve.” For a pigtail configuration, a flexible tension member, such as a suture thread, extends through draw ports at two spaced positions along the distal portion of the catheter. The restraining portion is conventionally activated by manually pulling the suture thread so that the two draw ports move toward each other as the pigtail loop forms at the distal end of the catheter. When the suture thread is taut, it prevents the pigtail loop from straightening by holding the juxtaposed portions of the catheter together in a locked position. The restraining portion is thus in a shape capable of resisting displacement from the body cavity. Once actuated, this restraining portion prevents removal of the catheter.

In percutaneous drainage procedures, the catheter is typically introduced into a patient by first introducing a hypodermic needle into the patient. A guidewire is inserted through the needle, which is then removed. The catheter tube is then passed over the previously emplaced guide wire into the drainage site in the body cavity. A stiffener in the form of a stiffening cannula or other implement may be inserted through the catheter lumen of the catheter tube to straighten the restraining portion of the catheter for introduction into the patient.

Currently, drainage catheters utilize a rigid, hollow plastic or metal stiffener that is inserted into the catheter to facilitate insertion. The tip of the stiffener engages with a shelf within the catheter lumen near the catheter tip, placing the catheter in tension so that the tip does not collapse during insertion. However, this tension creates a high degree of static friction (and/or compression) between the shelf of the catheter and the tip of the stiffener, which resists removal of the stiffener and can result in an undesirable removal stiffener force or sudden release of the stiffener which may result in undesirable movement of the catheter tip. In addition, if the force required to remove the stiffener is sufficiently high, it may result in breakage of the stiffener or require the need to remove the entire drainage assembly.

It would be desirable to provide a stiffener configuration that facilitates the reduction of friction between the stiffener tip and the catheter tip so that less force is required for removal of the stiffener from the catheter.

### SUMMARY

The devices, systems and methods described herein relate to percutaneous drainage catheters and a co-axial two-piece stiffener for use during catheter insertion and removal. The stiffener comprises inner and outer stiffener members with the inner member having a large diameter portion with an unconstrained diameter that is the same or larger than the diameter of the outer stiffener member and a reduced diameter when constrained by inner diameter of the outer stiffener member that is smaller than the diameter of the outer stiffener member.

In one embodiment, the large diameter portion comprises a flared distal portion with one or more relief slots or cuts longitudinally extending proximally from the distal end.

In another embodiment, the inner stiffener member is formed from a softer material than the outer stiffener member. The large diameter portion of the inner stiffener member includes a stepped portion to create a distal flared portion.

Other systems, methods, features and advantages will be or will become apparent to one with skill in the art upon examination of the following figures and detailed description. It is intended that all such additional systems, methods, features and advantages be included within this description, be within the scope of the devices, systems and methods described herein, and be protected by the accompanying claims.

### BRIEF DESCRIPTIONS OF THE DRAWINGS

The figures provided herein are not necessarily drawn to scale, with some components and features being exaggerated for clarity. Each of the figures diagrammatically illustrates aspects of the embodiments.

FIG. 1A is a schematic view of a catheter with a “pig tail” loop configuration as an anchoring mechanism, shown before the activation of the pig tail.

FIG. 1B is a schematic view of a catheter with a “pig tail” loop configuration as an anchoring mechanism, shown after the activation of the pig tail.

FIG. 2 is a partial cross-sectional view taken along line 2-2 in FIG. 1A depicting a straightened drainage catheter tip with conventional stiffener inserted in the catheter.

FIG. 3 is a perspective view depicting a distal portion of an exemplary embodiment of a stiffener.

FIG. 4 is a partial cross-sectional view depicting a straightened drainage catheter tip with the stiffener of FIG. 3 inserted in catheter.

FIG. 5 is a perspective view depicting a distal portion an alternative embodiment of a stiffener.

It should be noted that elements of similar structures or functions are generally represented by like reference numerals for illustrative purpose throughout the figures. It should also be noted that the figures are only intended to facilitate the description of the preferred embodiments.

### DETAILED DESCRIPTION

The devices, systems and methods described herein can be used for introducing a percutaneous catheter into a patient and anchoring the catheter into the body of the patient to facilitate draining fluid or removing other materials from the body. Alternatively, the catheter can introduce substances, such as fluids, into the patient's body.

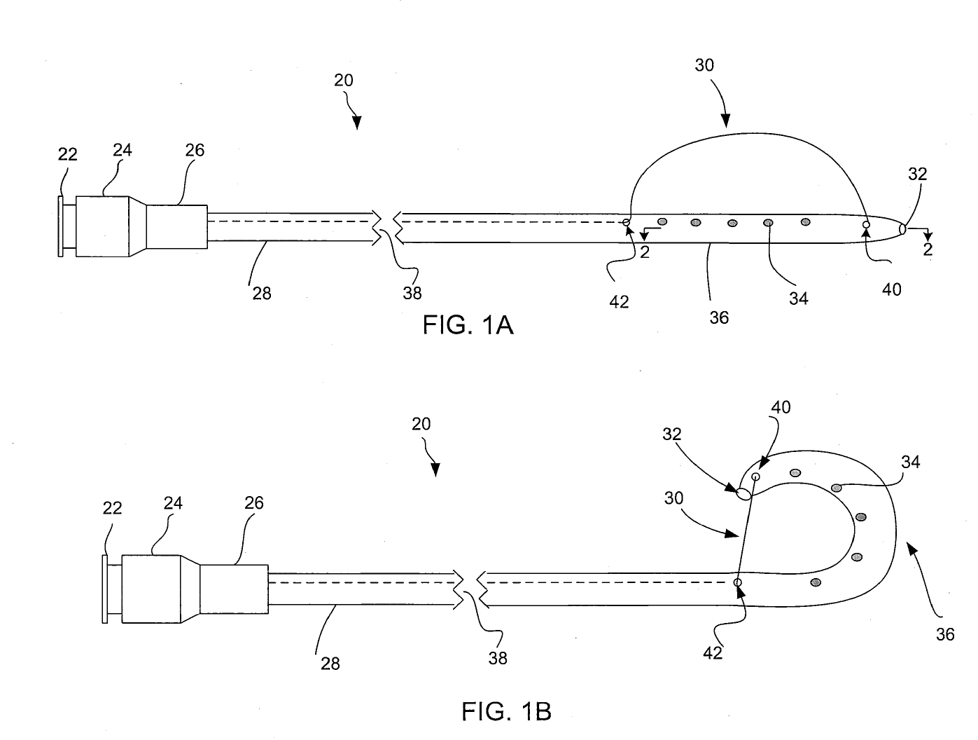

FIG. 1A depicts an example of a conventional drainage catheter 20. As depicted, the drainage catheter 20 comprises a flexible, elongate tube member 28 having a proximal end 22, a distal end 32, and a restraining portion 36. The wall of the restraining portion 36 toward the distal end 32 defines a series of drainage holes 34. The elongate tube member 28 defines an internal lumen 38, which extends through the catheter 20 and carries a flexible tension member 30, such as a suture thread. The tension member 30 extends through draw ports 40, 42 at two spaced positions on the restraining portion 36. The restraining portion 36 can be preformed into a “pigtail loop” shape from a shape-memory material or it can just extend along the longitudinal axis of the catheter 20. As shown in FIG. 1A, the restraining portion 36 extends along the horizontal axis. When the catheter 20 reaches the drainage site, the draw ports 40, 42 are drawn toward each other. As a result, the restraining portion 36 is formed into the shape of a pigtail, as shown in FIG. 1B. The pigtail loop configuration can be helped into place and secured by manipulating the tension member 30 at the proximal end 22 of the catheter, where the hub 24 is located. After the desired pigtail is formed, the tension member 30 is locked into position via a hub-locking mechanism 26 to maintain the distal pigtail shape.

Other restraining means utilizing a preformed curve as an anchoring mechanism are possible, such as a malecot rib fixation. In such a configuration, longitudinal slits are located in the restraining portion of the catheter, so that a malecot rib comprising of multiple wings is formed as the tension member is manipulated at the proximal end.

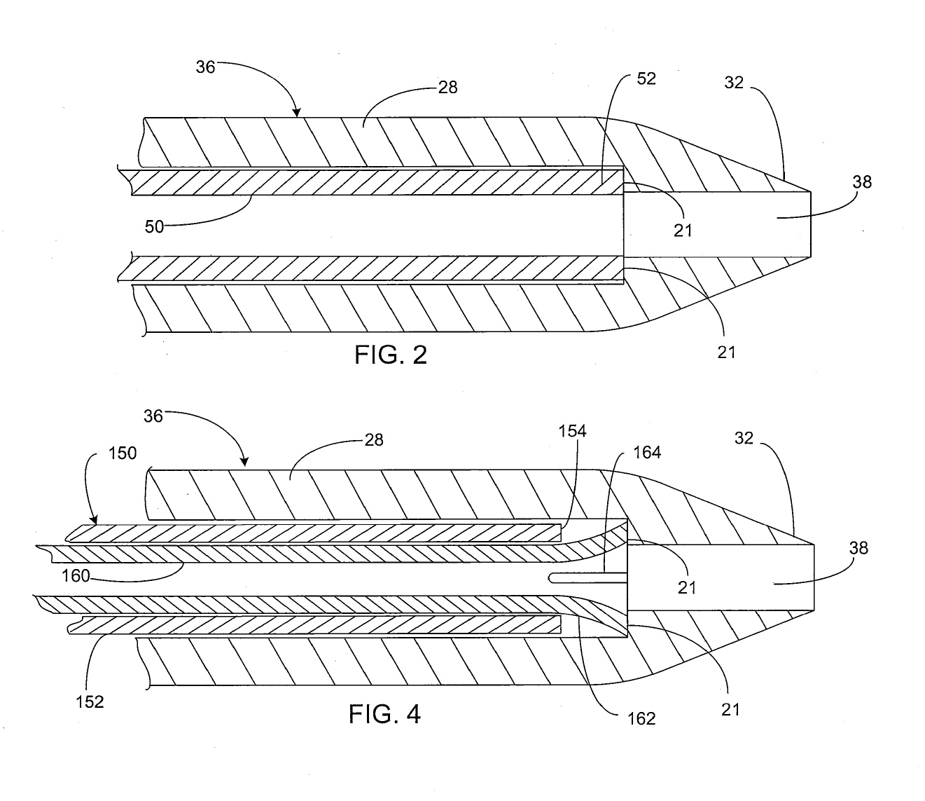

When the catheter 20 is first introduced into the patient, a stiffener, such as a stiffening cannula, can be inserted into the catheter lumen 38 to straighten the catheter 20. Referring to FIG. 2, a partial cross sectional view of a straightened retraining portion 36 of the drainage catheter 20 is depicted with a conventional stiffener 50 inserted in the lumen 38. The conventional stiffener 50 is typically an elongate rigid, hollow plastic or metal stiffening cannula that is inserted into the catheter 20 to facilitate insertion of the catheter 20 into a patient. The tip or distal end 52 of the stiffener 50 engages with a shelf 21 within the catheter lumen 38 near the distal end 32 of the catheter 20, placing the catheter 20 in tension so that the tip or restraining portion 36 does not collapse during insertion. However, this tension creates a high degree of static friction (and/or compression) between the shelf 21 of the catheter 20 and the tip 52 of the stiffener 50, which resists removal of the stiffener 50 and can result in an undesirable sudden release of the stiffener 50 and movement of the distal end 32 of the catheter 20. In addition, if the force required to remove the stiffener 50 is sufficiently high, it may result in breakage of the stiffener 50. It is desirable to reduce the friction between the stiffener tip 52 and the shelf 21 of the catheter 20 so that less force is required for removal of the stiffener 50.

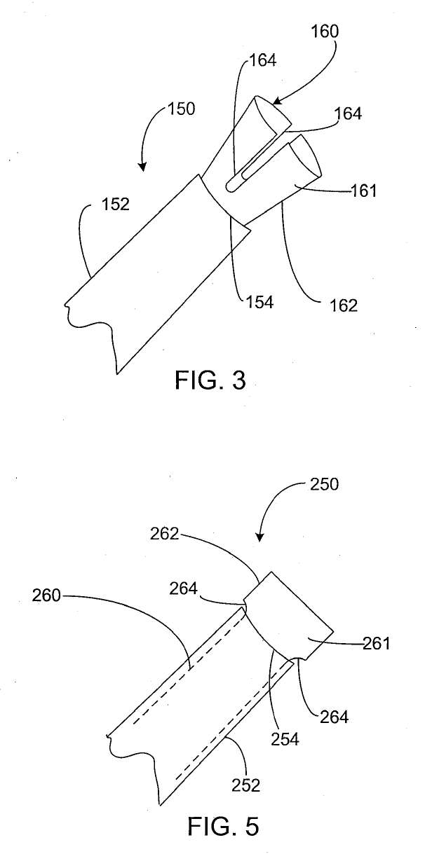

In a preferred embodiment shown in FIGS. 3 and 4, an elongate co-axial two-piece stiffener 150 comprises an outer stiffener component 152 preferably made of steel, nitinol or plastic, and an inner stiffener component 160 preferably made of steel, plastic or nitinol. The outer stiffener component 152 does not contact the shelf 21 of the catheter 20. The inner stiffener component 160 includes a flared tip or distal end 162 that contacts the shelf part 21 of the catheter 20, which creates contact and/or applies tension to the catheter 20. The flared tip or large diameter portion of the distal end 162 of the inner stiffener component 160 has an unconstrained diameter that is the same as or larger than the diameter of the outer stiffener component 152. To allow the inner stiffener component 160 to be inserted through and withdrawn from the outer stiffener component 152, the flared tip or large diameter portion of the distal end portion 162 of the inner stiffener component 160 comprises one or more, or more preferably two to four, relief cuts or slots 164 extending proximally from the distal end 162 of the inner stiffener component 160 so that the flared tip or large diameter portion 161 of the distal end 162 of the inner stiffener component 160 can collapse into a narrower diameter. The slits 164, as depicted, preferably extend parallel to the longitudinal axis of the inner stiffener component 160 or alternatively extend at an angle relative to the longitudinal axis of the inner stiffener component 160.

The configuration of the co-axial two-piece stiffener 150 is intended to decouple the compression, interference and friction forces acting upon the stiffener 150 by the catheter 20, which tend to retard withdrawal of a stiffener after placement. Upon removal or retraction of the inner stiffener component 160, the compression forces from the tip or distal end 32 of the catheter 20 acting on the stiffener 150 are removed or reduced. Subsequently, the removal of the outer stiffener component 152 has to primarily overcome friction forces to remove if from the catheter 20. By controlling or decoupling these forces, the potential for “stuck” stiffeners and or the risk of catheter tip displacement during removal of the stiffener is reduced.

In operation, the inner stiffener component 160 is inserted through the outer stiffener component 152 until the flared portion 161 extends beyond the distal end 154 of the outer stiffener component 152. The inner stiffener component 160 and outer stiffener component 152 are locked in position with the flared portion 161 extended beyond the distal end 154 of the outer stiffener component 152 via a threaded or similar locking mechanisms built into the proximal hubs of the inner stiffener component 160 and outer stiffener component 152.

The stiffener 150 is then inserted into the lumen 38 of the catheter 20 until the flared portion 162 contacts the shelf 21 of the catheter 20 placing the catheter 20 under tension and straightening the restraining portion 36 of the catheter 20. The catheter 20 and stiffener 150 assembly is then inserted over a guide wire to position the restraining portion 36 at the drain site within the patient. With the catheter 20 in position, the inner stiffener component 160 is drawn proximally relative to the outer stiffener component 152. As the inner stiffener component 160 is drawn proximally, the flared tip or large diameter portion 161 of the distal end 162 engages the distal end 154 of the outer stiffener component 152 causing the flared portion 162 to collapse along the slots 164 to a smaller diameter capable of passing into and through the lumen of the outer stiffener component 152. With the flared tip or large diameter portion 161 retracted into the lumen of the outer stiffener component 152 or the inner stiffener component 160 is removed entirely from the outer stiffener component 152, the compression/interference and friction forces acting on a stiffener 150 by the catheter 20 are decoupled or removed and the outer stiffener component 152 can be removed from the lumen 38 of the catheter 20.

Turning to FIG. 5, an alternative embodiment of a co-axial two piece stiffener 250 is shown. Rather than using a mechanical configuration to collapse the diameter of an inner stiffener 260, this embodiment of the stiffener 250 uses material properties of the of the inner stiffener member 260 to collapse it diameter. The inner stiffener member 260 is preferably made from a relatively soft material that can be collapsed into a relatively harder outer stiffener member 252 when drawn against the distal end 254 of the outer stiffener member 252. An example of this may be an outer stiffener member 252 constructed of stainless steel and an inner stiffener member 260 constructed of polyethylene which can deform to retract into or pass through the outer stiffener member 252. As depicted, the inner stiffener member 260 includes a stepped portion 264 to create the flared or large diameter portion 261 at the distal end 262 that contacts the shelf 21 in the catheter 20.

Exemplary embodiments, together with details regarding material selection and manufacture have been set forth above. As for other details of the presently described subject matter, these can be appreciated in connection with the above-referenced patents and publications as well as generally know or appreciated by those with skill in the art. The same can hold true with respect to method-based aspects in terms of additional acts as commonly or logically employed.

In addition, though the devices, systems and methods described herein have been presented herein in reference to exemplary embodiments, optionally incorporating various features, the devices, systems and methods described herein are not to be limited to that which is described or indicated as contemplated with respect to each variation. Various changes can be made to the subject matter described herein, and equivalents (whether recited herein or not included for the sake of some brevity) can be substituted without departing from the true spirit and scope of the disclosure.

It will be apparent to those skilled in the art that various modifications and variations can be made in the apparatus and method of the present invention without departing from the spirit or scope of the invention. Thus, it is intended that the present invention cover the modifications and variations of this invention provided they come within the scope of the appended claims and their equivalents.

1. A stiffener for use with an indwelling percutaneous catheter comprising:
an outer stiffener member, and
an inner stiffener member received in the outer stiffener member, the inner stiffener member having a large diameter portion at its distal end with an unconstrained diameter when positioned outside of the outer stiffener that is the same as or larger than the diameter of the outer stiffener member, wherein the large diameter portion is collapsible to a reduced diameter that is the same as or smaller than the inner diameter of the outer stiffener member.

2. The stiffener of claim 1 wherein the large diameter portion comprises a flared distal tip.

3. The stiffener of claim 2 wherein the flared distal tip comprising one or more proximally extending relief slots to allow the flared distal portion to collapse to a diameter that is the same as or smaller than the inner diameter of the outer stiffener member when drawn into and constrained by the outer stiffener member.

4. The stiffener of claim 1 wherein the flared distal tip comprising one or more proximally extending relief slots to allow the flared distal portion to collapse to a diameter that is the same as or smaller than the inner diameter of the outer stiffener member when drawn into and constrained by the outer stiffener member.

5. The stiffener of claim 1 wherein the outer stiffener member is formed from a first material and the inner stiffener member is formed from a second material that is softer than the first material.

6. The stiffener of claim 5 wherein the large diameter portion includes a stepped portion that creates a distal flared portion.

7. A method of inserting a drainage catheter into a patient comprising the steps of
inserting an elongate stiffener into the lumen of a drainage catheter until the distal end of the stiffener abuts a shelf in the lumen of the catheter, wherein the stiffener comprises an outer stiffener member and an inner stiffener member received in the outer stiffener member, the inner stiffener member having a large diameter portion at its distal end positioned outside of the outer stiffener and having a diameter that is the same as or larger than the diameter of the outer stiffener member,
inserting the drainage catheter with the stiffener inserted therein into a patient until the distal tip of the drainage catheter is positioned within a drain site within the patient,
drawing the inner stiffener member proximally relative to the outer stiffener member causing the large diameter portion to collapse to a reduced diameter that is the same as or smaller than the inner diameter of the outer stiffener member, and
withdrawing the inner and outer stiffener members from the lumen of the drainage catheter.

8. The method of claim 7 wherein the step of withdrawing the inner and outer stiffener members includes withdrawing the inner stiffener from the outer stiffener prior to withdrawal of the outer stiffener member from the lumen of the drainage catheter.

9. The method of claim 7 wherein the large diameter portion comprises a flared distal portion.

10. The method of claim 9 wherein the flared distal portion comprising one or more longitudinally extending slots to allow the flared distal portion to collapse to a diameter that is smaller than the diameter of the outer stiffener member when drawn into and constrained by the outer stiffener member.

11. The method of claim 7 wherein the large diameter portion comprising one or more proximally extending relief slots to allow the large diameter portion to collapse to diameter that is smaller than the diameter of the outer stiffener member when drawn into and constrained by the outer stiffener member.

12. The method of claim 7 wherein the outer stiffener member is formed from a first material and the inner stiffener member is formed from a second material that is softer than the first material.

13. The method of claim 7 wherein the large diameter portion includes a stepped portion that creates a distal flared portion.
